(19) United States
(12) Patent Application Publication
(10) Pub. No.: US 2020/0163734 A1
(43) Pub. Date: May 28, 2020

(54) MEDICAL INSTRUMENT DISPOSAL HOLDING AND VIEWING COMPARTMENT

(71) Applicant: BON SECOURS HEALTH SYSTEM, INC., Columbia, MD (US)

(72) Inventors: Jean Snyder, Columbia, MD (US); Nicole Marie Weikert, Columbia, MD (US); Nikolai Poulsen, Columbia, MD (US); Matthew Ibarra, Columbia, MD (US)

(21) Appl. No.: 16/632,324

(22) PCT Filed: Jul. 18, 2018
(86) PCT No.: PCT/US18/42639

§ 371 Date: Jan. 17, 2020

Related U.S. Application Data
(60) Provisional application No. 62/535,492, filed on Jul. 21, 2017.

(51) Int. Cl. A61B 50/36; B65F 1/10

(52) U.S. Cl. CPC A61B 50/362; A61B 2050/3008; B65F 1/10

(57) ABSTRACT

The present disclosure relates to a medical instrument holding container. The medical instrument holding container may comprise: a perimeter wall with at least one transparent portion; a holding shelf disposed within the perimeter wall, the holding shelf moveable between a resting position and an activated position; and a control mechanism coupled to the holding shelf and capable of being activated so as to move the holding shelf between the resting position and the activated position.

### CROSS-REFERENCE TO RELATED APPLICATIONS

This application claims priority to U.S. Provisional Application No. 62/535,492, entitled “SHARPS DISPOSAL HOLDING AND VIEWING COMPARTMENT”, filed on Jul. 21, 2017, the entirety of which is incorporated by reference herein in its entirety.

### TECHNICAL FIELD

The subject matter described herein relates to a holding and viewing compartment positioned adjacent a medical instrument disposal compartment.

### BACKGROUND

During a medical procedure, multiple medications and/or fluids can be delivered to a patient and multiple sharp devices (e.g., needles, syringe, scalpels, etc.) can be used. It is standard practice to dispose of some used medical related devices, such as medicinal vials and associated sharps, in a protective sharps disposal bin. Such sharps disposal bins are generally opaque, such as to protect patient information (e.g., on medicinal vial labels), and may not be emptied after each procedure. As such, it can be difficult for a medical professional to determine and cross-reference what medicaments or fluids have been delivered to a patient once the medical related devices have been disposed into the sharps disposal bin. This can result in medical errors (e.g., delivering too much or not enough of either a medicament or fluid) and/or prolonged procedure time. As such, there is a need for improved devices and methods for safely containing medical related devices after use.

### SUMMARY

A first aspect of the disclosure relates to a medical instrument holding container. The medical instrument holding container may include: a perimeter wall with at least one transparent portion; a holding shelf disposed within the perimeter wall, the holding shelf moveable between a resting position and an activated position; and a control mechanism coupled to the holding shelf and capable of being activated so as to move the holding shelf between the resting position and the activated position.

The medical instrument holding container may span a majority of the cross-sectional area of the medical instrument holding container. In the resting position, the holding shelf may be positioned to retain medical instruments within the medical instrument holding container. In the activated position, the holding shelf may be positioned such that the medical instruments are caused to be released from the holding shelf The control mechanism may extend through a portion of the perimeter wall such that activation of the control mechanism can cause movement of the holding shelf The control mechanism may include at least one of: a knob, a rod, a pin, a latch, a hinge, or a switch.

In some embodiments, the medical instrument holding container may further include a track disposed within a portion of the perimeter wall. The control mechanism may slidingly engage the track to cause movement of the holding shelf between the resting position and the activated position. The track may be stepped or angled.

In some embodiments, the medical instrument holding container may further include a first support coupled to the perimeter wall within the medical instrument holding container; and a second support coupled to the perimeter wall within the medical instrument holding container. A height of the first support may be greater than a height of the second support. In the resting position, one end of a bottom surface of the holding shelf may be engaged with the first support and an opposing, second end of the bottom surface of the holding shelf may be engaged with the second support. In the activated position, the first end of the bottom surface of the holding shelf may be disengaged from the first support and the second end of the bottom surface of the holding shelf may be disengaged from the second support.

In some embodiments, the holding shelf may include a flexible hinge. The activation of the control mechanism may cause the flexible hinge to bend thereby enabling the holding shelf to move between the resting position and the activated position. The control mechanism may bias the flexible hinge in a first position when the holding shelf is in the resting position.

In further embodiments, the medical instrument holding container may include a roller positioned within the medical instrument holding container and extending across a width of the medical instrument holding container such that one end of the holding shelf contacts the roller. In addition, a roller control mechanism may actively engage with the roller such that upon activation of the roller control mechanism, the roller rotates. In another embodiment, the holding shelf may be composed of a plurality of rollers, and a roller control mechanism may actively engage the holding shelf such that upon activation of the roller control mechanism, the plurality of rollers rotates.

The medical instrument holding container may include a guiding track in additional embodiments, the guiding track may extend through the perimeter wall. The guiding track may be at least one of: substantially curved or angled. The control mechanism may be coupled to the holding shelf through the guiding track. In such embodiments, the holding shelf may also be at least one of: substantially curved or angled.

In further embodiments, the control mechanism may include a rod extending at least partially through the perimeter wall such that a bottom surface of one end of the holding shelf engages the rod. The medical instrument holding container may also include a counter weight positioned about an opposing end of the holding shelf in some embodiments.

In further embodiments, the holding shelf may be composed of a plurality of panels capable of slidingly engaging one another upon activation of the control mechanism as the holding shelf moves between the resting position and the activated position. In yet a further embodiment, the holding shelf may include a pair of pivoting panels that are capable of pivoting downward.

Another aspect of the disclosure relates to a medical instrument disposal system. The system may include: a medical instrument disposal bin; a lid; and a medical instrument holding container releasably coupled to the medical instrument disposal bin and the lid such that the medical instrument holding container is disposed between the medical instrument disposal bin and the lid, wherein the medical instrument holding container includes: a perimeter wall with at least one transparent portion; a holding shelf disposed within the perimeter wall and spanning a majority of the cross-sectional area of the medical instrument holding container, the holding shelf moveable between a first, resting position and a second, activated position; and a control mechanism coupled to the holding shelf and capable of being activated so as to move the holding shelf between the resting position and the activated position, wherein, in the resting position, the holding shelf is positioned to retain medical instruments within the medical instrument holding container, and wherein, in the activated position, the holding shelf is positioned such that the medical instruments are caused to be released from the holding shelf and travel into the medical instrument disposal bin.

In some embodiments, the medical instrument disposal system may include a bracket substantially surrounding at least the medical instrument holding container and the medical instrument disposal bin. The bracket may include a sensor for identifying medical instruments positioned adjacent to the sensor. The sensor may include at least one of: a camera, a laser, a bar code scanner, an optical sensor, active light sensor, or load sensor. The sensor may be communicatively coupled to an electronic medical records system.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A shows a side view of an embodiment of a medical instrument holding container consistent with implementations of the current subject matter with a holding shelf of the sharps holding container in a first, resting position;

FIG. 1B shows the medical instrument holding container of FIG. 1A with the holding shelf in a second, activated position;

FIG. 2 illustrates a first embodiment of a track formed along the holding container of FIGS. 1A-1B for positioning the holding shelf in the resting and activated positions;

FIG. 3 illustrates a second embodiment of a track formed along the holding container of FIGS. 1A-1B for positioning the holding shelf in the resting and activated positions;

FIGS. 4A and 4B illustrate a side view of another embodiment of the holding container in a resting position and an activated position, respectively;

FIGS. 5A-5B illustrate a side view of another embodiment of the holding container in a resting position and an activated position, respectively;

FIGS. 6A-6B illustrate a side view of another embodiment of the holding container in a resting position and an activated position, respectively;

FIGS. 7A-7B illustrate a side view of another embodiment of the holding container in a resting position and an activated position, respectively;

FIGS. 8A-8B illustrate a side view of another embodiment of the holding container in a resting position and an activated position, respectively;

FIGS. 9A-9B illustrate a side view of another embodiment of the holding container in a resting position and an activated position, respectively;

FIGS. 10A-10B illustrate a side view of another embodiment of the holding container in a resting position and an activated position, respectively;

FIGS. 11A-11B illustrate a side view of another embodiment of the holding container in a resting position and an activated position, respectively;

FIGS. 12A-12B illustrate a side view of another embodiment of the holding container in a resting position and an activated position, respectively;

FIGS. 13A-13B illustrate a side view of another embodiment of the holding container in a resting position and an activated position, respectively;

14A-14B illustrate a side view of another embodiment of the holding container in a resting position and an activated position, respectively; and

FIG. 15 shows a perspective view of a medical instrument disposal system.

It is noted that the drawings of the subject matter are not necessarily to scale. The drawings are intended to depict only typical aspects of the subject matter, and therefore, should not be considered as limiting the scope of the disclosed subject matter. In the drawings, like numbering represents like elements between the drawings.

### DETAILED DESCRIPTION

The present disclosure describes various embodiments of a container that is configured to capture medical related instruments for viewing and dispose of such medical related instrument as a result of user interaction with the medical related instrument holding container, as will be described in greater detail below. As used herein, “medical related instrument” and/or “medical instrument” may refer to for example, any conventional medical instrument generally disposed of in a sharps disposal container, such as medicinal vials, needles, syringes, scalpels, etc. The medical instrument holding container can provide many benefits, including being adaptable to currently available sharps disposal containers and allowing viewing of medical related devices used during a procedure in a secure enclosed compartment that allows for easy disposal into a sharps disposal bin.

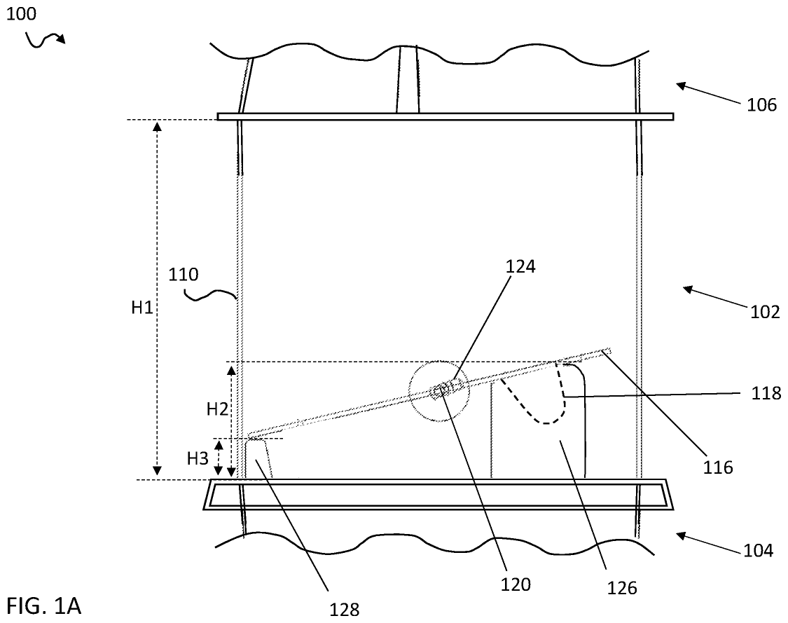

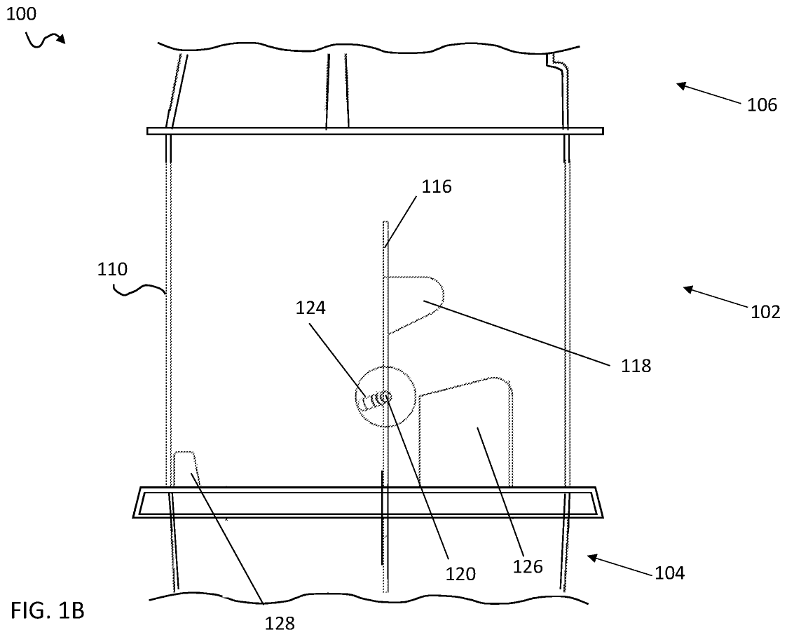

FIGS. 1A and 1B illustrate an embodiment of a medical instrument disposal assembly 100 including a medical instrument holding container 102. Medical instrument holding container 102 may be adaptable to currently available medical instrument or sharps disposal bin assemblies, which can include a medical instrument disposal bin 104 and a medical instrument lid 106. For example, medical instrument holding container 102 can have a bottom end that can be releasably coupled to medical instrument disposal bin 104 and a top end that can be releasably coupled to lid 106. In some embodiments, once the holding container 102 is coupled to lid 106, holding container 102 and lid 106 may be caused to be permanently coupled together and to disposal bin 104 such that medical instruments within assembly 100 cannot be removed therefrom. Holding container 102 described herein can be used with currently available sharps disposal bin assemblies 100, including either the currently available lids 106 or disposal bins 104. However, in some implementations, holding container 102 can be part of a medical instrument holding container assembly 100 that can include a lid 106 and/or disposal bin 104 such that holding container assembly 100 is integrated as one piece or part and can replace all or part of the currently available sharps disposal bin assemblies. In such an embodiment, entire assembly 100 can be disposed of once disposal bin 104 is full and/or according to conventional waste/sharps disposal practices. In additional implementations, holding container 102 can be integrated with lid 106 such that holding container 102 is an extension of lid 106 which can be coupled to disposal bin 104 through conventional means.

As shown in FIGS. 1A and 1B, holding container 102 can include a perimeter wall 110 with at least one portion (e.g., an entire side surface/sidewall of perimeter wall and/or a window within a side surface/sidewall) made of a transparent material that can allow a user to view the contents contained within holding container 102. In some embodiments, an entirety of perimeter wall 100 may be transparent. Holding container 102 can include a holding shelf 116 disposed within perimeter wall 110 that can position in at least a first, resting position (see, for example, FIG. 1A) and a second, activated position (see, for example, FIG. 1B). As used herein, the “resting position” may refer to the position of holding shelf 116 which can maintain or hold medical related instruments thereon within holding container 102, and the “activated position” may refer to the position of holding shelf 116 which causes the medical related instruments to be released from holding shelf 116 and travel towards or within disposal bin 104. In the resting position, holding shelf 116 can be flat or angled (e.g., approximately 5 degrees to 30 degrees) and configured to capture medical related devices that are disposed of through lid 106. In the angled configuration of the resting position, the medical related devices can be caused to travel towards perimeter wall 110, which can further assist in viewing the contents in holding container 102.

For example, in the resting position holding shelf 116 can be angled and span a majority of a cross-sectional area of holding container 102 (or more specifically, perimeter wall 110) thereby preventing medical related instruments to travel past holding shelf 116. This can allow the medical related instruments to collect on top of holding shelf 116 and be viewable by a user, such as during a medical procedure of a patient. As such, medical staff assisting with a procedure can easily see what has been delivered to or used on the patient while keeping the contents in a safe and enclosed container. In the activated position, holding shelf 116 can move, such as pivot, to allow the medical related instruments collected on the top of holding shelf 116 to fall through holding container 102 and into disposal bin 104. For example, this step can be performed once the procedure has been completed and proper documentation has been made regarding at least all delivered medicaments and fluids delivered to the patient.

After one or more dispenses of medical related instruments into disposal bin 104, disposal bin 104 can be decoupled from the bottom of holding container 102 and properly disposed of or emptied. An empty disposal bin 104 can then be recoupled to the holding container 102, thereby allowing holding container 102 to be reused multiple times. Holding container 102 can be made of a material that can allow holding container 102 to be cleaned and sterilized. Lid 106 can also be removed from holding container 102 for cleaning or disposal with disposal bin 104 and replaced with either lid 106 or holding container 102. In alternative embodiments, the entirety of disposal assembly 100 can be disposed of together.

As shown in FIGS. 1A and 1B, holding container 102 can include at least one control mechanism 120 (e.g., a knob, pin, rod, latch, hinge, and/or switch) that is coupled to holding shelf 116 and can be manipulated or activated by the user, so as to move holding shelf 116 between the resting and activated positions. For example, control mechanism 120 can extend through a part of perimeter wall 110, e.g., via an aperture (not shown) within perimeter wall 110, such that movement and/or rotation of control mechanism 120 can cause movement and/or rotation of holding shelf 116. In this way, holding shelf 116 is coupled within holding container 102 via control mechanism 120. In some embodiments, perimeter wall 110 can have a height H1 (or total length extending between bin 104 and lid 102) of approximately 5 inches tall or greater to allow for sufficient room to collect and view contents therein. However, various other sizes and shapes of perimeter wall 110 is within the scope of this disclosure. As used herein, the terms “about,” “substantially,” “approximately,” and variations thereof are intended to include the degree of error associated with measurement of the particular quantity based upon the equipment available at the time of filing the application.

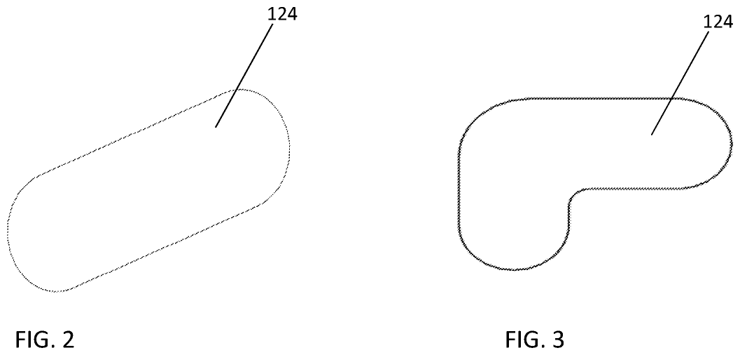

FIGS. 2 and 3 illustrate different embodiments of a track 124 that can be disposed within a portion perimeter wall 110 of holding container 102 (FIGS. 1A-1B). Track 124 can extend through the perimeter wall 110 to allow control mechanism 120 to extend therethrough. Track 124 can be configured such that control mechanism 120 can travel or slide therealong to move holding shelf 116 between the resting and activated positions (see FIGS. 1A-1B). For example, as shown in FIG. 2, track 124 can be angled (e.g., approximately 5 degrees to approximately 30 degrees) such that placement of control mechanism 120 in a lower position (relative to the angle of track 124) causes holding shelf 116 to position in the resting position (FIG. 1A), and placement of control mechanism 124 in an upper position (relative to the angle of track 124) causes holding shelf 116 to position in the activated position (FIG. 1B). As shown in FIG. 3, in another embodiment, track 124 can be stepped (e.g., joined horizontal and vertical segments) such that placement of control mechanism 120 in lower vertical position (relative to the steps of track 124 in FIG. 3) causes holding shelf 116 to position in the resting position (FIG. 1A) and placement of control mechanism 120 in the end horizontal position (relative to the steps of track 124 in FIG. 3) causes holding shelf 116 to position in the activated position (FIG. 1B). Various features associated with holding container 102 can assist with moving holding shelf 116 between the resting and activated positions.

For example, as shown in FIGS. 1A-1B, holding container 102 can include at least a first support 126 and at least a second support 128 coupled to, affixed to, and/or integrated with at least a portion of an internal surface of perimeter wall 110. In some embodiments, one support 126, 128 can be taller or have a greater height than the other support 126, 128. Referring to FIGS. 1A-1B, height H2 of first support 126 is shown as being greater than height H3 of second support 128. Such a configuration can promote holding shelf 116 to form an angle when holding shelf 116 is resting on first and second supports 126, 128 together. Supports 126, 128 can be shaped and positioned within sharps holding container 102 such that, in the resting position, the bottom surface of holding shelf 116 is resting on a top surface of both supports 126, 128. More specifically, the bottom surface of a first end of holding shelf 116 may engage with first support 126 and the bottom surface of the opposing end of holding shelf 116 may engage with second support 128 in the resting position. In an optional embodiment, holding shelf 116 may include an increased weight and/or weighted feature 118 (shown behind support 126 in the side view of FIG. 1A) integrated with and/or attached thereto that biases holding shelf 116 against one or more supports 126, 128, and further biases holding shelf 116 in the resting position. When holding shelf 116 is moved to the activated position, holding shelf 116 can slide off or disengage from supports 126, 128 upon activation of control mechanism 120 and due to the weight of any medical instruments positioned on holding shelf 116. This can allow holding shelf 116 to pivot into the activated position at least because of the medical related devices or instruments resting on the top surface of holding shelf 116 causing holding shelf 116 to pivot into an approximately vertical position (e.g., the activated position). Furthermore, in some embodiments, the user can rotate control mechanism 120 to control the pivoting of holding shelf 116, including to reset holding shelf 116 into the resting position. In an optional embodiment, where weighted feature 118 is included, weighted feature 118 may also assist in returning holding shelf 116 to the resting position. The shape of tracks 124 can be configured to allow the user to control and/or manipulate holding shelf 116 between resting and activated positions while also preventing unintended movement of control mechanism 120. Both the angled track and the stepped track of FIGS. 2 and 3, respectively, encourage control mechanism 120 to be in the first, lower position and require a user to force control mechanism 120 into the second position, such as an angled upward direction or sequentially vertically up and horizontal directions. In alternative embodiments, supports 126, 128 may be approximately the same height, but may be positioned at different heights about perimeter wall 110. Various other configurations of track 124 and/or supports 126, 128 are within the scope of this disclosure.

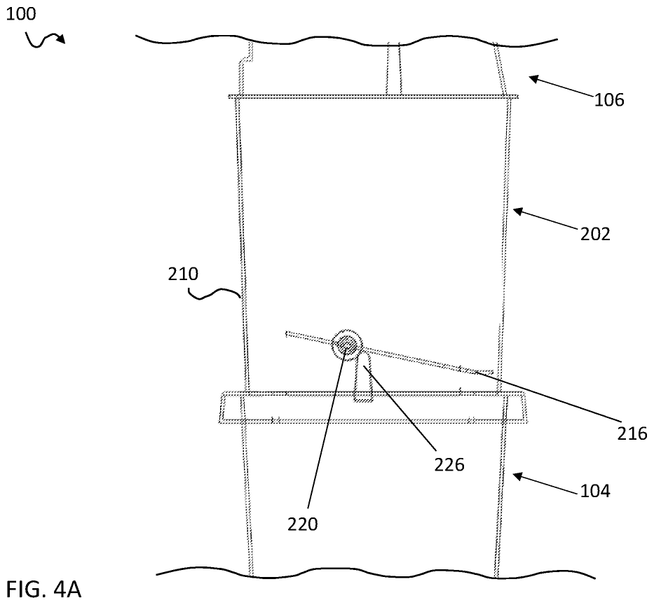

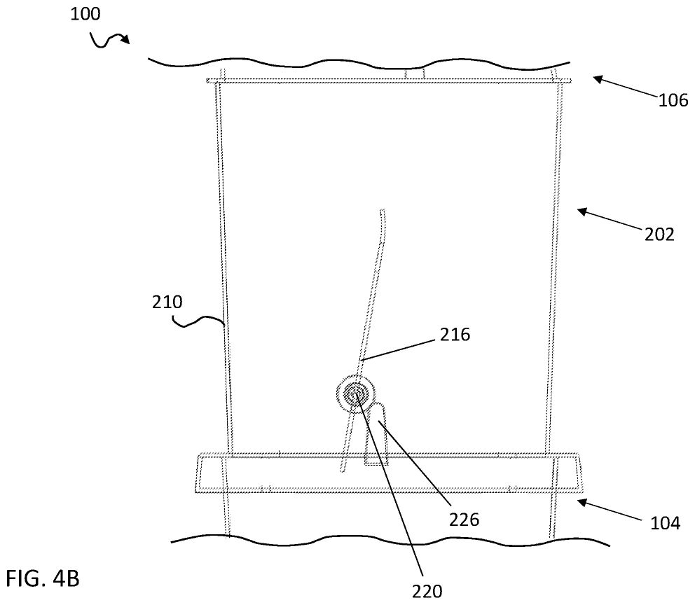

FIGS. 4A-4B illustrate a second embodiment of a holding container 202 that is similar to holding container 102 of FIGS. 1A and 1B regarding adaptability to currently available medical instrument or sharps disposal assemblies and including a perimeter wall 210 with at least a part that is transparent for allowing viewing therethrough into holding container 202. As shown in FIGS. 4A-4B, this may include an entire side surface/sidewall of perimeter wall 210, however, other locations about perimeter wall 210 are also possible. Holding container 202 may also include a holding shelf 216 that is moveable between a first, resting position (shown in FIG. 4A) and a second, activated position (shown in FIG. 4B). Holding shelf 216 can be angled and span a majority of a cross-sectional area of sharps holding container 202 (or more specifically, perimeter wall 210) thereby preventing medical related instruments to travel past holding shelf 216.

Holding container 202 can also include a control mechanism 220 that may be coupled to holding shelf 216, and can be manipulated by a user to cause holding shelf 216 to move between the resting and activated positions. For example, control mechanism 220 can be rotated by a user to cause holding shelf 202 to pivot between the resting and activated positions. Control mechanism 220 can extend at least partially through perimeter wall 210 to couple to holding shelf 216. In this way, holding shelf 216 is coupled and maintained within holding container 202 via control mechanism 220. In addition, as shown in FIGS. 4A-4B, holding container 202 can include a support 226 for supporting/assisting in maintaining holding shelf 216 in the resting position. Like supports 126, 128 of FIGS. 1A-1B, support 226 may be coupled to, affixed to, and/or integrated with at least a portion of perimeter wall 110. In this way, a bottom surface of holding shelf 216 may contact support 226 and rest at least partially on support 226 in the resting position.

As shown in FIG. 4A, in the resting position, holding shelf 216 can be angled such that one end of holding shelf 216 is positioned lower than another end of holding shelf 216.

Support 226 may assist in maintaining a downward angle of holding shelf 216 such that a lower end of holding shelf 216 is proximate an internal surface of at least one side of perimeter wall 210. Support 226 can also prevent holding shelf 216 from pivoting further such as to cause medical related instruments from traveling into disposal bin 104. In an optional embodiment, lower end of holding shelf 216 can include an increased weight or weighted feature (not individually shown in FIGS. 4A-4B, but similar to weighted feature 118 of FIGS. 1A-1B) that biases holding shelf 216 against support 226, and further biases holding shelf 216 in the resting position. The dispending part of lid 106 can be positioned relative to holding shelf 216 such that medical related instruments or contents that are dispensed into holding container 102 are caused to collect towards the lower end of holding shelf 216. When desired, such as after completion of a medical procedure, a user can rotate or activate control mechanism 220 to cause the lower end of holding shelf 216 to pivot upward into the activated position (FIG. 4B) thereby causing the medical related contents to slide off the opposing end of holding shelf 216 and into disposal bin 104.

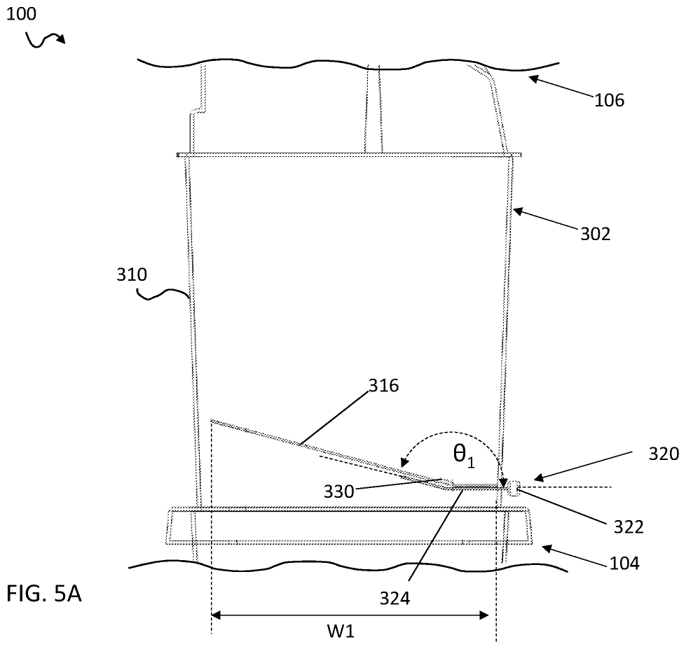

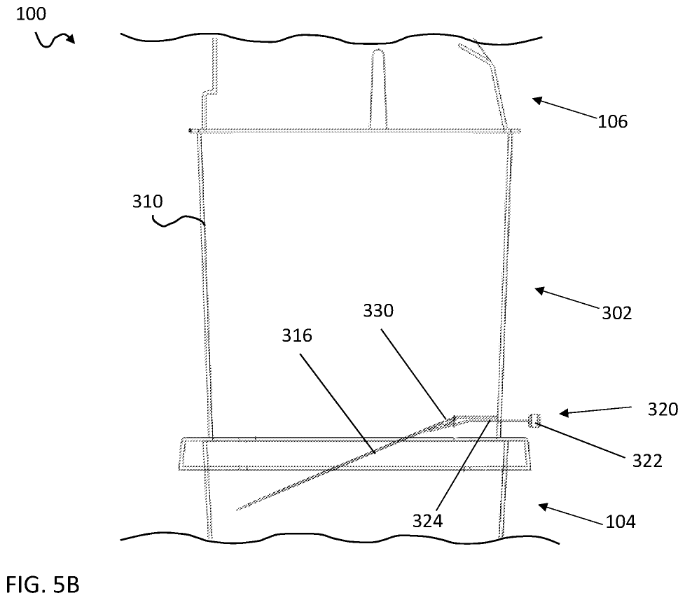

FIGS. 5A-14B illustrate additional example embodiments of a holding container including different variations of a holding shelf For example, FIGS. 5A-5B illustrate a holding container 302 with a perimeter wall 310 and a holding shelf 316. Like perimeter wall 110 of holding container 102 (FIGS. 1A-1B), perimeter wall 310 may include at least one transparent portion to enable viewing inside holding container 302. As shown in FIGS. 5A-5B, this may include an entire side surface/sidewall of perimeter wall 310, however, other locations about perimeter wall 310 are also possible. Holding shelf 316 can be angled and span a majority of a cross-sectional area of sharps holding container 302 (or more specifically, perimeter wall 310) thereby preventing medical related devices to travel past holding shelf 316. In this embodiment, holding shelf 316 may further include a flexible hinge (or living hinge) 330 positioned about a width W1 of holding shelf 316 that allows holding shelf 316 to bend at flexible hinge 330 to transition from the resting position (as shown in FIG. 5A) to the activated position (e.g., holding shelf 316 angled downward to allow contents on holding shelf 316 to fall into disposal bin 104 as shown in FIG. 5B) upon activation of control mechanism 320. More specifically, flexible hinge 330 may extend along an entirety of a length of holding shelf 316 (or into the page of FIG. 5A). Holding shelf 316 may maintain its position within holding container 302 by being secured (i.e., adhered, molded into, or fastened) directly to an internal surface of perimeter wall 310 on an end of holding shelf 316 that is nearest control mechanism 320. Thus, a free end of holding shelf 316 includes an end opposing the secured end which can alter its position based on the interaction of control mechanism 320 and flexible hinge 330 as will be discussed herein.

In this embodiment, for example, control mechanism 320 can include a knob 322 on the outside of perimeter wall 322 connected to or integrated with a rod 324 which may extend at least partially through perimeter wall 310. As shown, rod 324 may be positioned at or about flexible hinge 330. Rod 324 may provide support to and/or bias flexible hinge 330 thereby maintaining holding shelf 316 in the resting position and preventing travel of medical instruments past holding shelf 316. More specifically, a bottom surface of holding shelf 316 (including flexible hinge 330) may substantially rest on rod 324. As shown in FIG. 5A, rod 324 can have an angle θᵢ positioned about the length of rod 324 that is consistent with a desired angle of the position of holding shelf 316 in the resting position. In this way, a free end of holding shelf 316 is positioned at a desired angle due to holding shelf 316 being supported by and resting on rod 324. As previously discussed herein, the angled configuration of holding shelf 316 in the resting position allows any medical related instruments positioned thereon to be caused to travel towards perimeter wall 310, which can further assist in viewing the contents in sharps holding container 302. Control mechanism 320 (or more specifically knob 322) can be activated (e.g., pulled and/or rotated) to cause flexible hinge 330 to bend thereby enabling holding shelf 316 to move between the resting position and activated position. As shown in FIG. 5B, the pulling and/or rotating of knob 322 causes movement of rod 424 in such a way that rod 324 no longer provides support to flexible hinge 330 and causes pivoting of flexible hinge 330, thereby transitioning holding shelf 316 from the resting position to the activated position. In some embodiments, control mechanism 320 can be spring loaded to force control mechanism 320 back towards sharps holding container 302 when not being manipulated by a user, thereby resetting holding shelf 316 in the resting position.

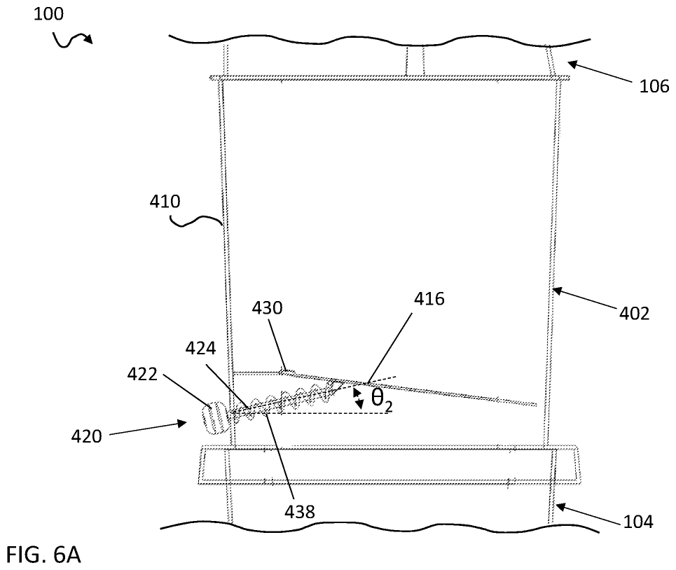

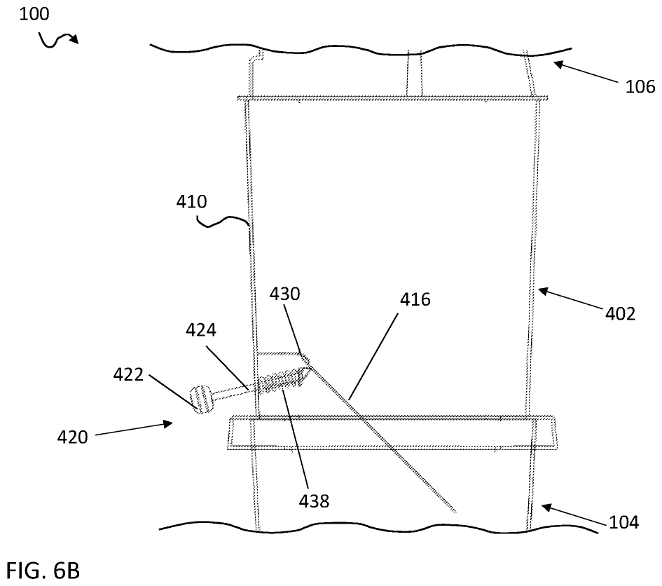

FIGS. 6A-6B illustrate another embodiment of holding container 402 similar to holding container 302 (FIGS. 5A-5B). Holding container 402 in this embodiment may include a perimeter wall 410 and a holding shelf 416. Like perimeter wall 110 of holding container 102 (FIGS. 1A-1B), perimeter wall 410 may include at least one transparent portion to enable viewing inside holding container 402. As shown in FIGS. 6A-6B, this may include an entire side surface/sidewall of perimeter wall 410, however, other locations about perimeter wall 410 are also possible. Holding shelf 416 can be angled and span a majority of a cross-sectional area of holding container 402 (or more specifically, perimeter wall 410) thereby preventing medical related instruments to travel past holding shelf 416. One end of holding shelf 416 may be coupled to perimeter wall 410. In this embodiment, holding shelf 416 may further include a flexible hinge (or living hinge) 430 positioned about a length of holding shelf 416 that allows holding shelf 416 to bend at flexible hinge 430 to transition from the resting position (as shown in FIG. 6A) to the activated position (e.g., holding shelf 416 angled downward to allow contents on holding shelf 416 to fall into disposal bin as shown in FIG. 6B). Holding shelf 416 may maintain its position within holding container 402 by being secured (i.e., adhered, molded into, or fastened) directly to an internal surface of perimeter wall 410 on an end of holding shelf 416 that is nearest control mechanism 420.

Thus, a free end of holding shelf 416 includes an end opposing the secured end which can alter its position based on the interaction of control mechanism 420 and flexible hinge 430 as will be discussed herein. In this embodiment, control mechanism 420 can include a knob 422 on the outside of perimeter wall 422 connected to or integrated with a rod 424 which may extend through perimeter wall 410. Rod 424 may provide support to holding shelf 416 thereby maintaining holding shelf 416 in the resting position and preventing travel of medical instruments past holding shelf 416. As shown, an end of rod 424 may contact a bottom surface of holding shelf 416 that is on an opposing side of flexible hinge 430 relative to the end of holding shelf 416 that is coupled to perimeter wall 410. More specifically, a bottom surface of holding shelf 416 may substantially rest on rod 424. However, any position of rod 424 capable of maintaining holding shelf 416 in the resting position is possible. As shown in FIG. 6A, rod 424 may extend through perimeter wall 410 at an angle 02 that enables positioning of holding shelf 416 at a desired angle in the resting position. In this way, holding shelf 416 is positioned at an angle due to holding shelf 416 being supported by and resting on rod 424. As previously discussed herein, the angled configuration of holding shelf 416 in the resting position allows any medical related devices positioned thereon to be caused to travel towards perimeter wall 410, which can further assist in viewing the contents in holding container 402. Control mechanism 420 (or more specifically knob 422) can be pulled and/or rotated to allow holding shelf 416 to position in the activated position (FIG. 6B). As shown in FIG. 6B, the pulling of knob 422 causes movement of rod 424 in such a way that causes pivoting of flexible hinge 430 such that holding shelf 416 transitions from the resting position to the activated position. In this way, the pulling of knob 422 provides less support for holding shelf 416 which causes flexible hinge 430 to pivot due to the weight of holding shelf 416. In some embodiments, control mechanism 420 can be spring loaded, e.g., via a spring 438 substantially surrounding rod 424, to force control mechanism 420 back towards holding container 402 when not being manipulated by a user, thereby resetting holding shelf 416 in the resting position (FIG. 6A).

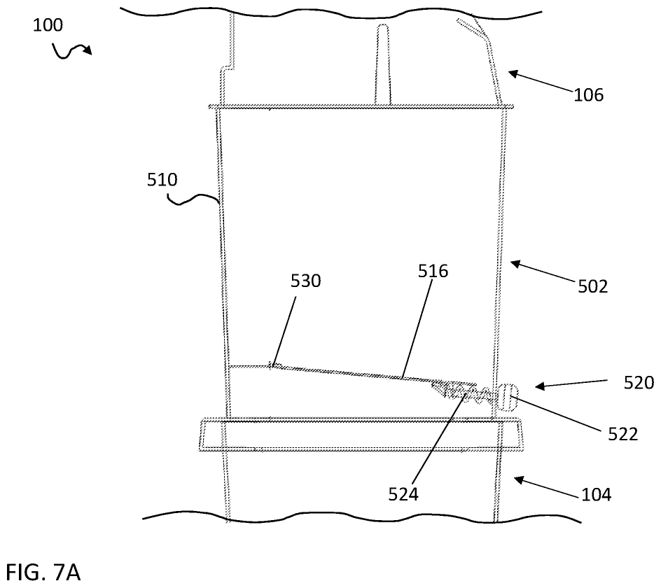

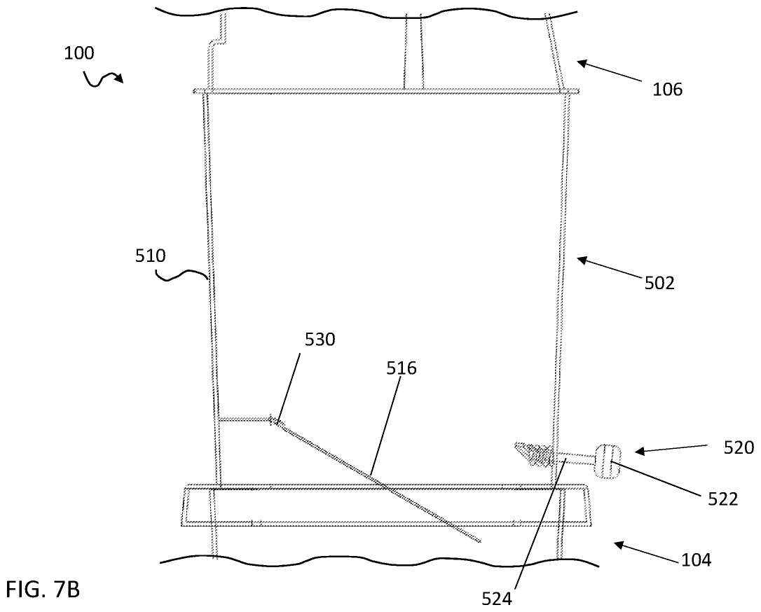

FIGS. 7A-7B illustrate another embodiment of holding container 502 similar to holding container 402 (FIGS. 6A-6B). Holding container 502 in this embodiment may include a perimeter wall 510 and a holding shelf 516. Like perimeter wall 110 of sharps holding container 102 (FIGS. 1A-1B), perimeter wall 510 may include at least one transparent portion to enable viewing inside holding container 502. As shown in FIGS. 7A-7B, this may include an entire side surface/sidewall of perimeter wall 510, however, other locations about perimeter wall 510 are also possible. Holding shelf 516 can be angled and span a majority of a cross-sectional area of holding container 502 (or more specifically, perimeter wall 510) thereby preventing medical related instruments to travel past holding shelf 516. One end of holding shelf 516 may be coupled to perimeter wall 510. In this embodiment, holding shelf 516 may further include a flexible hinge (or living hinge) 530 positioned about a length of holding shelf 516 that allows holding shelf 516 to bend at flexible hinge 530 to transition from the resting position (as shown in FIG. 7A) to the activated position (e.g., holding shelf 516 angled downward to allow contents on holding shelf 516 to fall into disposal bin as shown in FIG. 5B). Holding shelf 516 may maintain its position within holding container 502 by being secured (i.e., adhered, molded into, or fastened) directly to an internal surface of perimeter wall 510 on an end of holding shelf 516 that is furthest control mechanism 520, or on an end of holding shelf 516 that opposes the end of holding shelf 516 that interacts with control mechanism 520. Thus, a free end of holding shelf 516 includes an end opposing the secured end which is capable of altering its position based on the interaction of control mechanism 520 and flexible hinge 530 as will be discussed herein.

In this embodiment, control mechanism 520 can include a knob 522 on the outside of perimeter wall 522 connected to or integrated with a rod 524 which may extend at least partially through perimeter wall 510. Rod 524 may provide support to holding shelf 516 thereby maintaining holding shelf 516 in the resting position and preventing travel of medical instruments past holding shelf 516. As shown, an end of rod 524 may contact a bottom surface of holding shelf 516 that is on an opposing side of flexible hinge 530 relative to the end of holding shelf 516 coupled to perimeter wall 510. More specifically, a bottom surface of holding shelf 516 may substantially rest on rod 524. However, any position of rod 524 capable of maintaining holding shelf 516 in the resting position is possible. As shown in FIG. 7A, rod 524 may extend through perimeter wall 510 at an angle 02 that enables positioning of holding shelf 516 at an angle in the resting position. In this way, holding shelf 516 is positioned at an angle due to holding shelf 516 being supported by and resting on rod 524. As previously discussed herein, the angled configuration of holding shelf 516 in the resting position allows any medical related instruments positioned thereon to be caused to travel towards perimeter wall 510, which can further assist in viewing the contents in holding container 502. Control mechanism 520 (or more specifically knob 522) can be pulled and/or rotated to allow holding shelf 516 to position in the activated position (FIG. 7B). As shown in FIG. 7B, the pulling of knob 522 causes movement of rod 524 in such a way that causes pivoting of flexible hinge 530 such that holding shelf 516 transitions from the resting position to the activated position. In this way, the pulling of knob 522 provides less support for holding shelf 516 which causes flexible hinge 530 to pivot due to the weight of holding shelf 516. In some embodiments, holding shelf 516 can be light enough and flexible hinge 530 can be resilient/stiff enough to spring back into the resting position after the contents on holding shelf 516 have been disposed into disposal bin 104. Control mechanism 520 can be released after holding shelf 516 has returned to the resting position to thereby secure holding shelf 516 in the resting position. In some embodiments, control mechanism 520 can be spring loaded, e.g., via a spring 538 substantially surrounding rod 524, to force control mechanism 520 back towards holding container 502 when not being manipulated by a user, thereby resetting holding shelf 516 in the resting position (FIG. 7A).

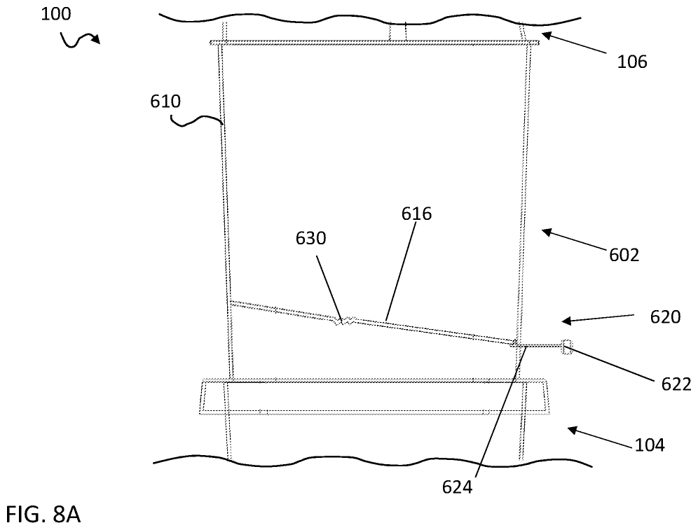

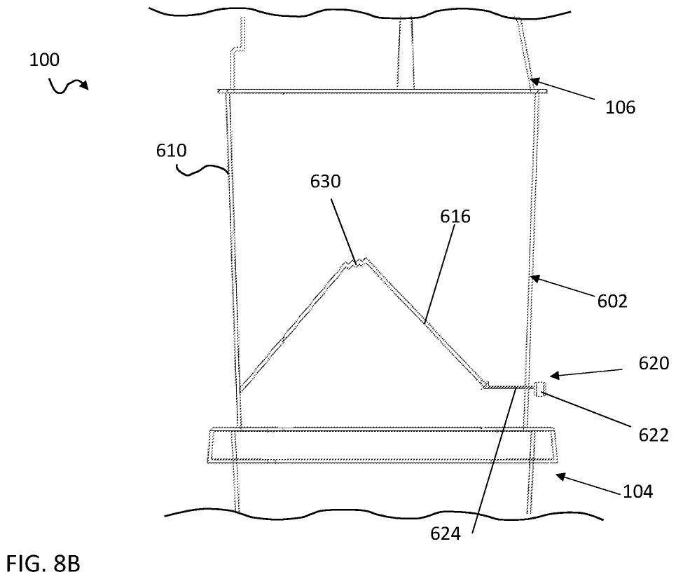

FIGS. 8A-8B illustrate another embodiment of a holding container 602 similar to the holding container (502 (FIGS. 7A-7B) of the previous embodiments such that holding container 602 includes perimeter wall 610 having at least one transparent portion, a holding shelf 616, and a flexible hinge 630 positioned along a length of holding shelf 616. Holding shelf 616 may maintain its position within holding container 602 by being secured (i.e., adhered, molded into, or fastened) directly to an internal surface of perimeter wall 610 on an end of holding shelf 616 that is furthest control mechanism 620, or on an end of holding shelf 616 that opposes the end of holding shelf 616 that interacts with control mechanism 620. Thus, a free end of holding shelf 616 includes an end opposing the secured end which is capable of altering its position based on the interact of control mechanism 620 and flexible hinge 630 as will be discussed herein. In some embodiments, a second flexible hinge 630 may be used to secure holding shelf 616 within perimeter wall 610 at the secured end of holding shelf 616.

In this embodiment, control mechanism 620 can include a knob 622 on the outside of perimeter wall 622 connected to or integrated with a rod 624 which may extend through perimeter wall 610. Rod 624 may provide support to holding shelf 616 thereby maintaining holding shelf 616 in the resting position and preventing travel of medical instruments past holding shelf 616. As shown, an end of rod 624 within perimeter wall 610 may contact an end and/or a bottom surface of holding shelf 616 that is on an opposing side of flexible hinge 630 relative to the end of holding shelf 616 coupled to perimeter wall 610. Holding shelf 616 may be positioned at an angle within perimeter wall 610 such that holding shelf 616, in the resting position, allows any medical related instruments positioned thereon to be caused to travel towards perimeter wall 610, which can further assist in viewing the contents in holding container 602. Control mechanism 620 (or more specifically knob 622) can be pushed to allow holding shelf 616 to position in the activated position (FIG. 8B). As shown in FIG. 8B, the pushing of knob 622 causes movement of rod 624 in such a way that causes pivoting of flexible hinge 630 in an upward direction such that holding shelf 616 transitions from the resting position to the activated position. In this way, the pushing of knob 622 causes flexible hinge 530 to pivot or fold upward due to the force of rod 624. In such an embodiment, medical instruments positioned on holding shelf 616 may slide off holding shelf 616 on opposing sides of rod 624 to be disposed of within disposal bin 104. After disposal of medical instruments, control mechanism 620 can be released thereby causing holding shelf 616 to return to the resting position to secure holding shelf 616 in the resting position. In some embodiments, control mechanism 620 can be spring loaded, e.g., via a spring (not individually shown) substantially surrounding rod 624 on the outside of perimeter wall 610, to force knob 622 away from perimeter wall 610 when not being manipulated by a user, thereby resetting holding shelf 616 in the resting position (FIG. 8A).

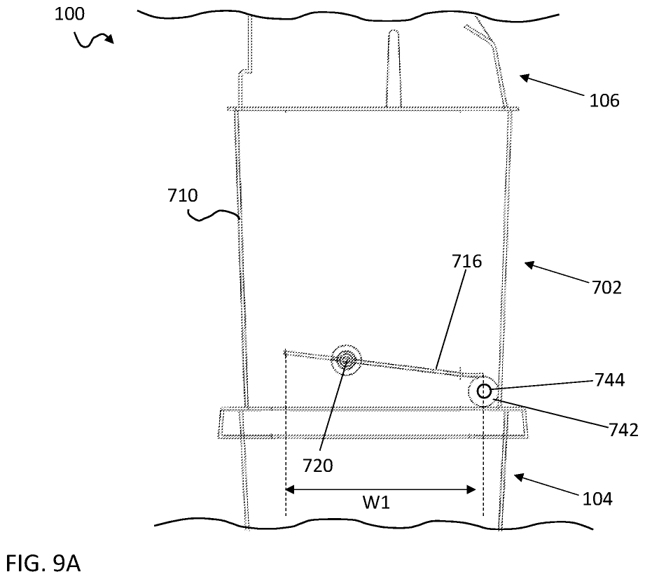

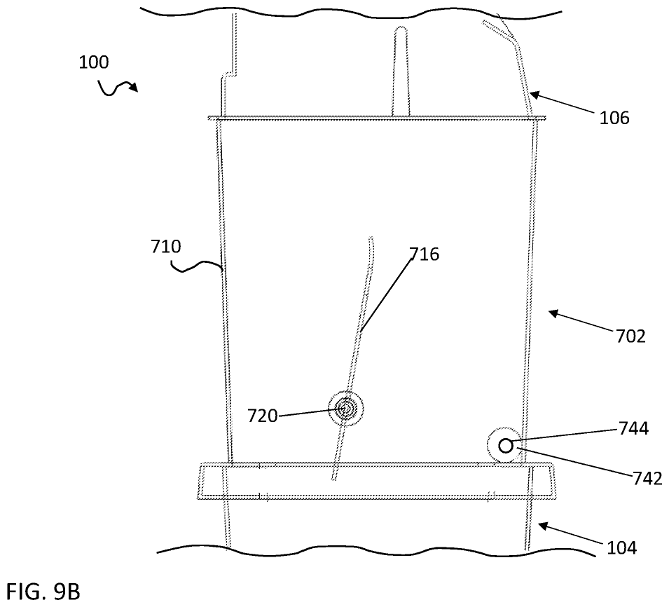

Turning now to FIGS. 9A-9B, another embodiment of a holding container 702 is shown. This embodiment may be similar to holding container 102 of FIGS. 1A-1B. Holding container 702 may include a perimeter wall 710 and a holding shelf 716 within perimeter wall 710, and spanning a majority of the cross-sectional area of perimeter wall 710 thereby preventing medical related instruments thereon from traveling into disposal bin 104. As shown, holding shelf 716 can be angled within perimeter wall 710 to aid in viewing medical related instruments on holding shelf 716 in the resting position. Holding container 702 can also include at least one control mechanism 720 that can be coupled to holding shelf 716 and can be manipulated by the user, such as to move holding shelf 716 between the resting and activated positions. For example, control mechanism 720 can extend at least partially through a part of perimeter wall 710 about a width W1 of holding shelf 716, e.g., via an aperture (not shown) within perimeter wall 710, such that movement and/or rotation of control mechanism 720 can cause movement or rotation of holding shelf 716. In this way, holding shelf 716 may be coupled within holding container 702 via control mechanism 720. As shown, control mechanism 720 can be rotated counterclockwise to pivot holding shelf 716 into the activated position (FIG. 9B) and rotated clockwise to return holding shelf 716 to the resting position (9A). Holding container 702 in this embodiment can further include a roller or rolling feature 742 and a roller control mechanism 744 (e.g., a knob) coupled to and/or actively engaged with roller 742 for controlling rotation of roller 742 when roller control mechanism 744 is manipulated or activated by the user. Roller 742 may extend across a width of holding shelf 716 and/or holding container 702. In some embodiments, a bottom surface of holding shelf 716 may rest on or contact roller 742. Roller 742 can, when rotated by roller control mechanism 744, cause contents or medical related instruments positioned on holding shelf 716 adjacent roller 742 to rotate. This can allow for better control of viewing of the contents within holding container 702, such as for reading label information on such contents.

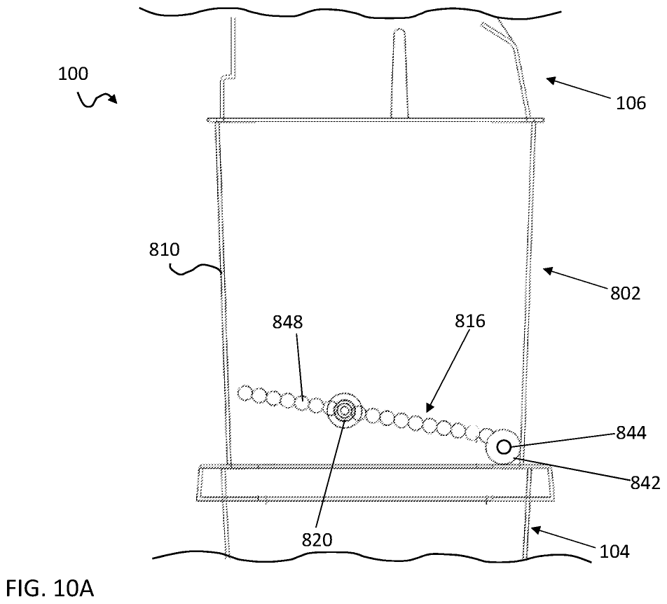

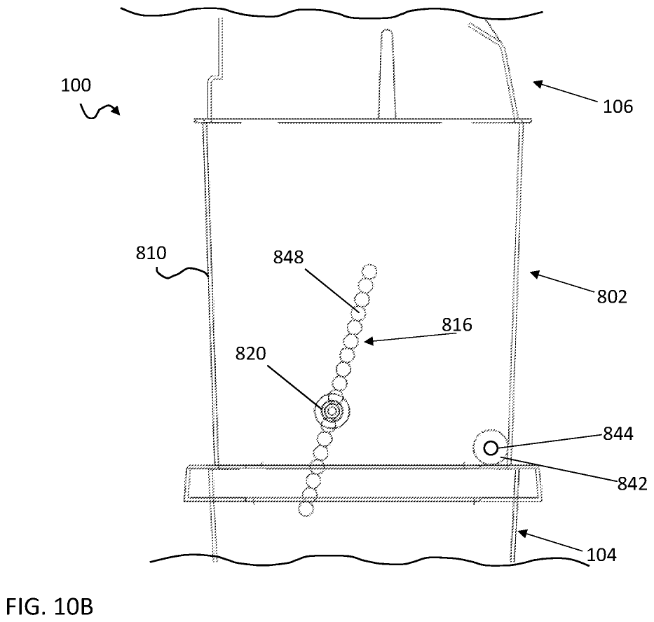

FIGS. 10A-10B illustrate another embodiment of a holding container 802. This embodiment is similar to that of holding container 702 (FIGS. 9A-9B) in that holding container 802 can include perimeter wall 810, holding shelf 816, control mechanism 820, roller 842, and roller control mechanism 844. However, in this embodiment, holding shelf 816 can be composed of a plurality of rollers 848 which may each individually rotate upon rotation of roller control mechanism 844. In alternative embodiments (not shown), the plurality of rollers 848 may be positioned along a length of holding shelf 816. Holding shelf 816 can pivot or rotate upon rotation of control mechanism 820. For example, rotation of roller control mechanism 844 may cause rotation of roller 842, which may in turn cause rotation of individual plurality of rollers 848 to cause medical related instruments positioned thereon to rotate to aid in viewing the medical related instruments. More specifically, in some embodiments, plurality of rollers 848 may be in contact each other just enough such that activation of roller control mechanism 844 may cause rotation of a first roller within plurality of rollers 848 that is nearest roller control mechanism 844. The rotation of the first roller may then cause rotation of the adjacent roller within plurality of rollers 848 (and so on) due to the rollers within plurality of rollers 848 being in contact with each other. Subsequently, control mechanism 820 can be rotated counterclockwise to pivot holding shelf 816 to the activated position and rotated clockwise to return holding shelf 816 into the resting position.

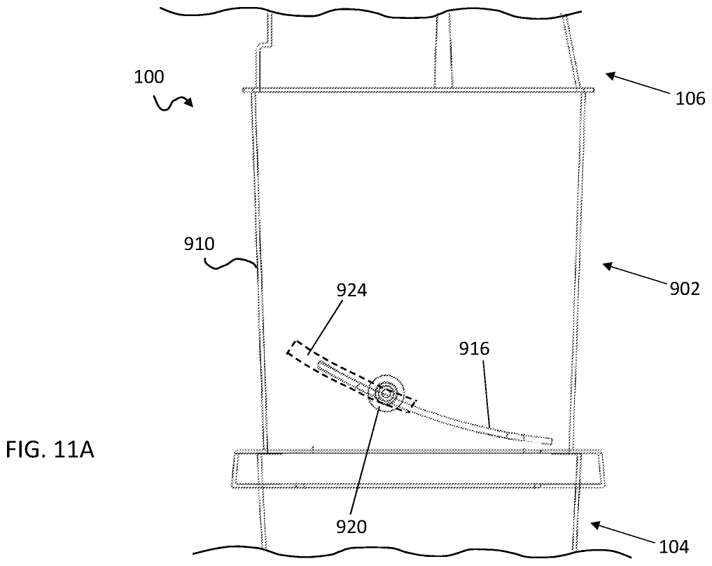

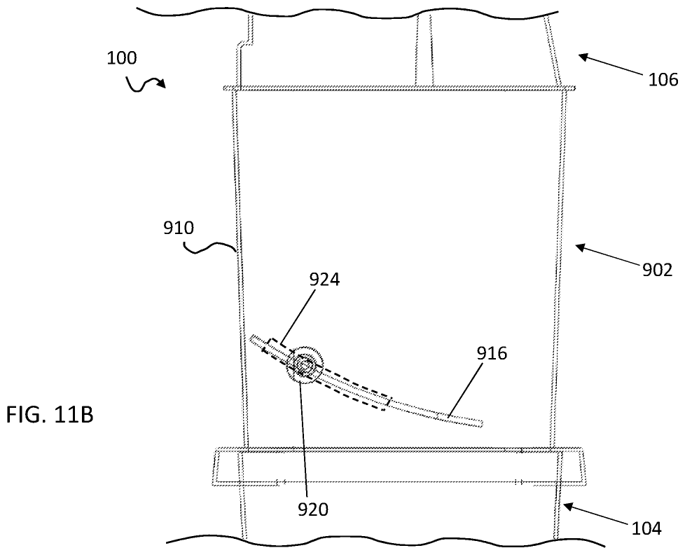

FIGS. 11A-11B illustrate another embodiment of holding container 902. This embodiment is similar to that of holding container 102 (FIGS. 1A-1B) in that holding container 902 can include perimeter wall 910, holding shelf 916, control mechanism 920, and a guiding track 924. Track 924 may extend through perimeter wall 910 and be substantially curved and/or angled. Control mechanism 920 may be coupled to holding shelf 916 through track 924. A user may manipulate control mechanism 920 such that control mechanism 920 translates about track 924 to cause holding shelf 916 to move between the resting position (FIG. 11A) and the activated position (FIG. 11B) to allow contents on holding shelf 916 to fall into disposal bin 104. In this embodiment, holding shelf 916 may be substantially curved and/or angled. Optionally, the curve and/or angle of holding shelf 916 may be substantially consistent with the curve and/or angle of track 924.

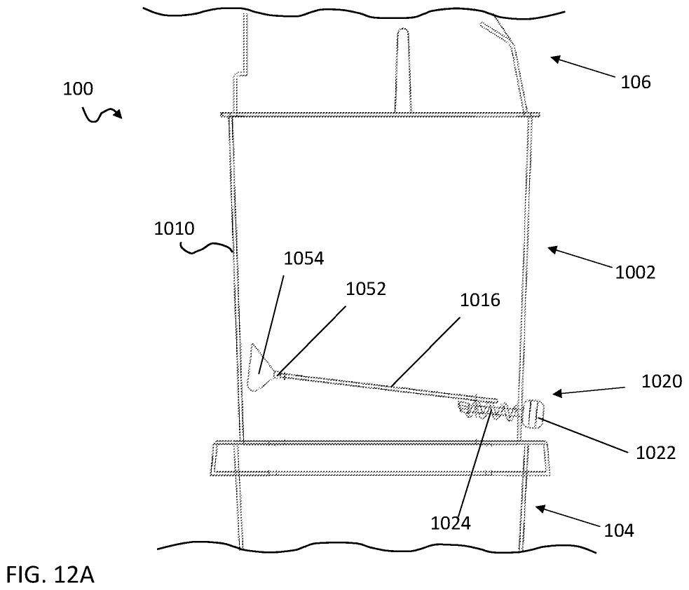

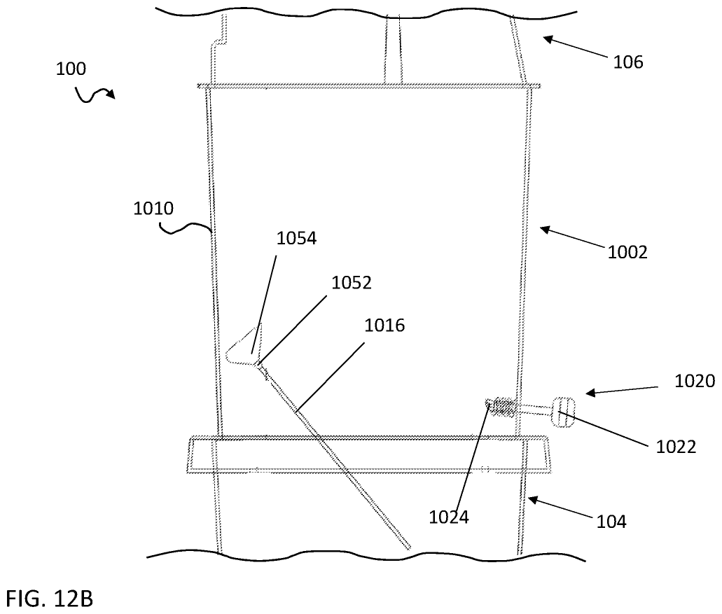

FIGS. 12A-12B illustrate another embodiment of holding container 1002. Holding container 1002 may include perimeter wall 1010, holding shelf 1016, and control mechanism 1020 having knob 1022 and rod 1024 similar to holding container 502 (FIGS. 7A-7B). Like control mechanism 520, knob 1022 may be positioned outside perimeter wall 1010 and rod 1024 may extend from knob 1022 at least partially through perimeter wall 1010 such that one end of a bottom surface of holding shelf 1016 rests on or engages rod 1024 within the resting position (FIG. 12A). However, in this embodiment, holding shelf 1016 further includes a pivot 1052 and a counter weight 1054. Pivot 1052 may include a pin extending through perimeter wall 1010 about which holding shelf 1016 pivots or rotates. Counter weight 1054 may be integrated with and/or attached to an end of holding shelf 1016 that is opposite the end of holding shelf 1016 supported by or engaged with rod 1024. Knob 1022 can be pulled to release holding shelf 1016 to the activated position. Counter weight 1054 can be light enough to allow holding shelf 1016, with medical related instruments resting thereon, to move into the activated position to allow the medical related instruments to fall into disposal bin 104. Additionally, counter weight 1054 can be heavy enough to cause holding shelf 1016 to spring or return back into the resting position after the medical related instruments on holding shelf 1016 have been disposed of into disposal bin 104. Knob 1022 can be released after holding shelf 1016 has returned to the resting position to thereby secure holding shelf 1016 in the resting position.

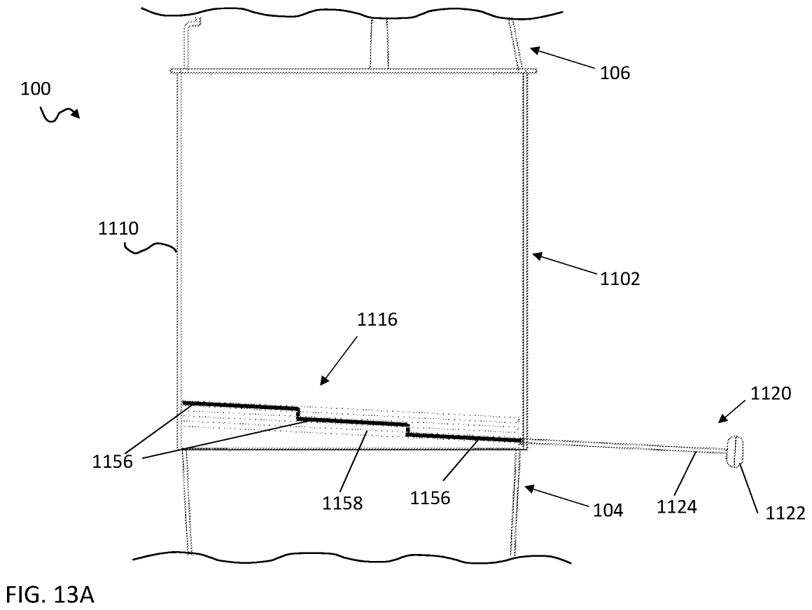

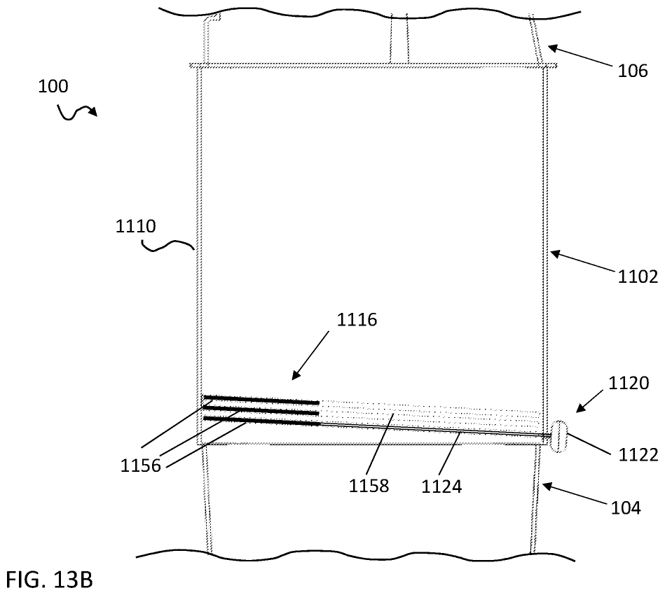

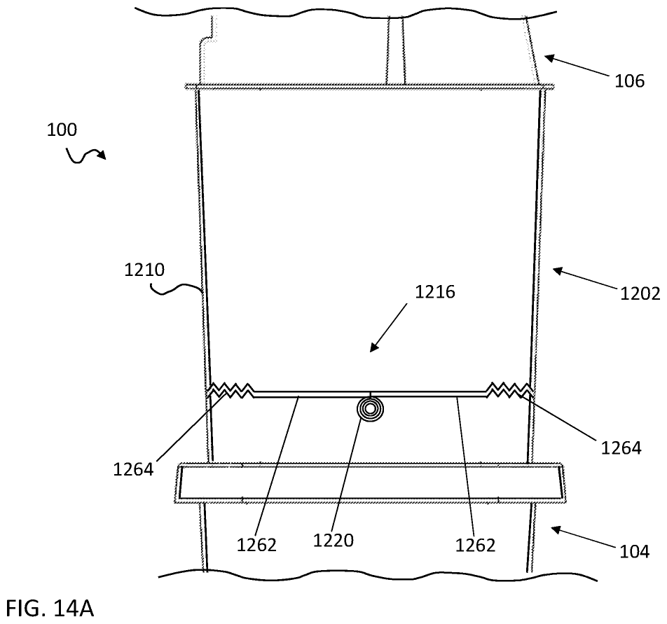

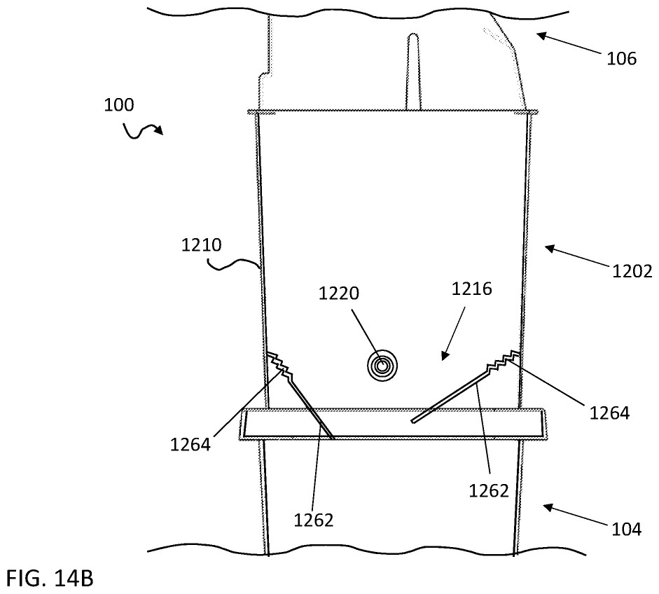

FIGS. 13A-13B illustrate another embodiment of holding container 1102. In this embodiment, holding container 1102 may include perimeter wall 1110 including at least one transparent portion, a holding shelf 1116, a control mechanism 1120 having a knob 1122 and a rod 1124. However, in this embodiment, holding shelf 1116 may be composed of a plurality of panels 1156 in sliding engagement with one another. Control mechanism 1120 can be actively coupled to holding shelf 1116 such that control mechanism 1120 (or more specifically, knob 1122) can be pushed to cause the plurality of panels 1156 to slide relative to one another to stack upon each other (e.g., the front panel slides under the second panel, and so forth) to thereby move the holding shelf 1116 into the activated position (FIG. 13B) and allow medical related contents resting on holding shelf 1116 to fall into disposal bin 104. After disposal of contents, control mechanism 1120 can be pulled to return holding shelf 1116 to the resting position. In an optional embodiment, perimeter wall 1110 may include guiding tracks 1158 therein for assisting with sliding the plurality of panels 1156.

FIGS. 14A-14B illustrate another embodiment of a holding container 1202. In this embodiment, holding container 1202 may include perimeter wall 1210 including at least one transparent portion, a holding shelf 1216, a control mechanism 1220 having a knob 1222 and a rod 1224. However, in this embodiment, holding shelf 1216 may be composed of a pair of oppositely facing, pivoting panels 1262, each including a flexible hinge 1264. Flexible hinges 1264 of panels 1262 may be coupled to, affixed to, and/or integrated with at least a portion of an internal surface of perimeter wall 1210. Panels 1262 can be caused to move between the resting (FIG. 14A) and activated (FIG. 14B) positions by control mechanism 1220 and/or flexible hinges 1264. In the activated position, the pair of panels 1262 can pivot downward to allow medical related contents held on the holding shelf 1216 to fall into disposal bin 104. Control mechanism 1220 can extend through perimeter wall 1210 and at least partially within holding container 1202. A free end of panels 1264 (an end of panels 1261 that is opposite flexible hinge 1264 secured to perimeter wall 1210) may at least partially rest on or engage a portion of control mechanism 1220 that extends within perimeter wall 1210 such that pulling of control mechanism 1220 by a user causes panels 1262 to release and transition to the activated position (FIG. 14B). Flexible hinges 1262 may be resilient/stiff enough to spring back into the resting position (FIG. 14A), and control mechanism 1220 can be pushed such that free end of panels 1262 engage control mechanism 1220 again, and remain in the resting position.

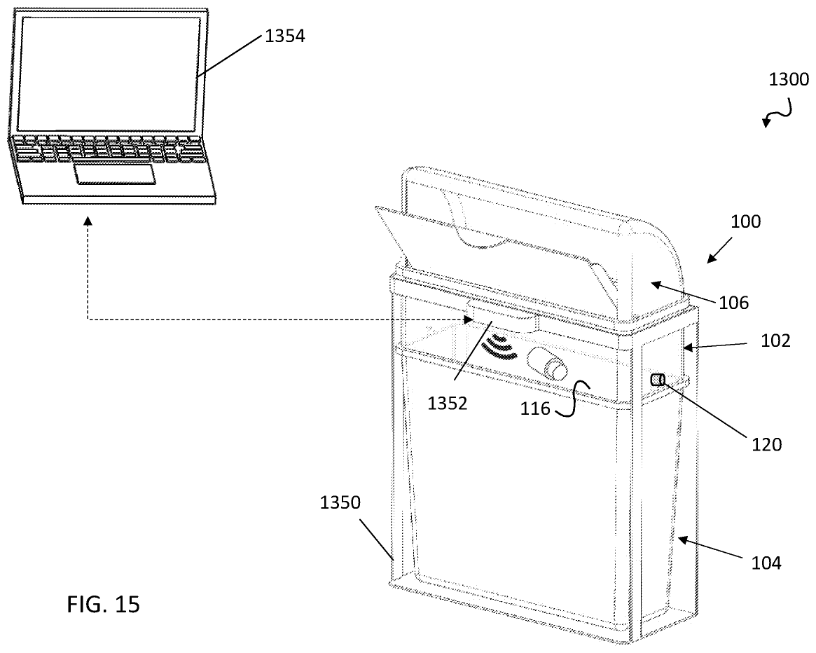

FIG. 15 shows a medical instrument disposal system 1300 including medical instrument disposal assembly 100 according to the embodiment of FIGS. 1A-1B. Further, medical instrument disposal system 1300 may include a bracket 1350 substantially surrounding medical instrument disposal assembly 100. More specifically, bracket 1350 may substantially surround holding container 102 and disposal bin 104. In some embodiments, bracket 1350 may assist in securing medical instrument disposal assembly 100 to a wall, counter, or cart within a medical procedure room, or some other fixture in a medical procedure room. In such an embodiment, bracket 1350 may be directly secured to the wall or counter via any conventional attachment means, such as, for example, bolts, screws, adhesives, etc. Bracket 1350 may include at least a closed bottom end and an open top end. Medical instrument disposal assembly 100 may slide in and out of bracket 1350 through the open top end and rest on the closed bottom end. While bracket 1350 is shown as being used with disposal assembly 100 including holding container 102 according to the embodiments of FIGS. 1A-1B, it is to be understood that bracket 1350 can be used with disposal assembly 100 having a holding container according to any embodiments discussed herein.

Bracket 1350 may include a sensor 1352 for identifying medical instruments positioned on holding shelf within holding container. Sensor 1352 can include at least one of: a camera, a laser, a bar code scanner, an optical sensor, active light sensor, or load sensor. Sensor 1352 may be used to further assist in identifying which medical instruments or medications were used with a patient during a medical procedure. Sensor 1352 may be communicatively coupled to a processor or computing system. For example, sensor 1352 may be communicatively coupled to an electronic medical records (EMR) system 1354 such that identification of the medical instruments and/or medications used during a procedure and disposed of in disposal system 1300 are communicated to the EMR 1354. Sensor 1352 may be communicatively coupled to EMR system 1354 with or without wires, i.e., wired or wirelessly.

Referring back to FIG. 1, another aspect of the disclosure relates to a method. The method may include dispensing at least one medical instrument through and/or within lid 106 of a medical instrument disposal assembly 100, and holding or retaining the at least one medical instrument on a holding shelf 116 within holding container 102 of assembly 100. Further, the method can include viewing or identifying the at least one medical instrument on holding shelf 116 within holding container 102, and moving holding shelf 116 from a resting position to an activated position thereby causing the at least one medical instrument to be released from holding shelf 116 and travel within a disposal bin 104 of assembly 100. In some embodiments, the at least one medical instrument may be viewed through a transparent portion of a perimeter wall 110 of holding container 102. Further, in some embodiments, moving holding shelf 116 between the resting and activated positions may include activating control mechanism 130 that is coupled to holding shelf 116. Once a medical procedure is complete and/or disposal bin 104 is full, the method can also include disposing of disposal bin 104 and/or assembly 100. As should be clear, any of the configurations discussed herein relative to holding shelves and/or control mechanisms may be used to perform the moving of the holding shelf and/or activating of the control mechanism.

The medical instrument holding container discussed herein can provide many benefits, including being adaptable to currently available sharps disposal containers and allowing viewing and identification of medical related devices used during a procedure in a secure enclosed compartment that allows for easy disposal into a medical instrument and/or sharps disposal bin. It is to be understood that embodiments of the holding container discussed herein may be equally applicable to other disposal bins aside from medical instrument and/or sharps disposal bins.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. Other configurations of the holding shelf are within the scope of this disclosure for allowing medical related devices to collect thereon for viewing by a user and moveable, such as by a control mechanism, to thereby allow the medical related instruments to be disposed of in disposal bin. In addition, the logic flows depicted in the accompanying figures and/or described herein do not necessarily require the particular order shown, or sequential order, to achieve desirable results. The scope of the following claims may include other implementations or embodiments.

What is claimed is:

1. A medical instrument holding container comprising:
a perimeter wall with at least one transparent portion;
a holding shelf disposed within the perimeter wall, the holding shelf moveable between a resting position and an activated position; and
a control mechanism coupled to the holding shelf and capable of being activated so as to move the holding shelf between the resting position and the activated position.

2. The medical instrument holding container of claim 1, wherein the holding shelf spans a majority of a cross-sectional area of the medical instrument holding container.

3. The medical instrument holding container of claim 1, wherein the control mechanism extends through a portion of the perimeter wall such that activation of the control mechanism can cause movement of the holding shelf.

4. The medical instrument holding container of claim 1, wherein, in the resting position, the holding shelf is positioned to retain medical instruments within the medical instrument holding container, and wherein, in the activated position, the holding shelf is positioned such that the medical instruments are caused to be released from the holding shelf.

5. The medical instrument holding container of claim 1, wherein the control mechanism includes at least one of a knob, a rod, a pin, a latch, a hinge, or a switch.

6. The medical instrument holding container of claim 1, further comprising:
a track disposed within a portion of the perimeter wall,
wherein the control mechanism slidingly engages the track to cause movement of the holding shelf between the resting position and the activated position.

7. The medical instrument holding container of claim 6, wherein the track is stepped or angled.

8. The medical instrument holding container of claim 1, further comprising:
a first support coupled to the perimeter wall within the medical instrument holding container; and
a second support coupled to the perimeter wall within the medical instrument holding container,
wherein a height of the first support is greater than a height of the second support and wherein a first end of a bottom surface of the holding shelf is engaged with the first support in the resting position and an opposing, second end of the bottom surface of the holding shelf is engaged with the second support in the resting position, and
wherein the first end of the bottom surface of the holding shelf is disengaged from the first support in the activated position and the second end of the bottom surface of the holding shelf is disengaged from the second support in the activated position.

9. The medical instrument holding container of claim 1, wherein the holding shelf includes a flexible hinge, and wherein activation of the control mechanism causes the flexible hinge to bend thereby enabling the holding shelf to move between the resting position and the activated position.

10. The medical instrument holding container of claim 11, wherein the control mechanism biases the flexible hinge in a first position when the holding shelf is in the resting position.

11. The medical instrument holding container of claim 1, further comprising:
a roller positioned within the medical instrument holding container and extending across a width of the medical instrument holding container such that one end of the holding shelf contacts the roller; and
a roller control mechanism actively engaged with the roller such that upon activation of the roller control mechanism, the roller rotates.

12. The medical instrument holding container of claim 1, wherein the holding shelf is composed of a plurality of rollers, and further comprising:
a roller control mechanism actively engaged with the holding shelf such that upon activation of the roller control mechanism, the plurality of rollers rotates.

13. The medical instrument holding container of claim 1, further comprising:
a guiding track extending through the perimeter wall, the guiding track being at least one of: substantially curved or angled,
wherein the control mechanism is coupled to the holding shelf through the guiding track, and wherein the holding shelf is at least one of: substantially curved or angled.

14. The medical instrument holding container of claim 1, when control mechanism includes a rod extending at least partially through the perimeter wall such that a bottom surface of one end of the holding shelf engages the rod, and further comprising:
a counter weight positioned about an opposing end of the holding shelf.

15. The medical instrument holding container of claim 1, wherein the holding shelf is composed of a plurality of panels capable of slidingly engaging one another upon activation of the control mechanism as the holding shelf moves between the resting position and the activated position.

16. The medical instrument holding container of claim 1, wherein the holding shelf includes a pair of pivoting panels that are capable of pivoting downward.

17. A medical instrument disposal system comprising:
a medical instrument disposal bin;
a lid; and
a medical instrument holding container releasably coupled to the medical instrument disposal bin and the lid such that the medical instrument holding container is disposed between the medical instrument disposal bin and the lid, wherein the medical instrument holding container includes:
a perimeter wall with at least one transparent portion;
a holding shelf disposed within the perimeter wall and spanning a majority of the cross-sectional area of the medical instrument holding container, the holding shelf moveable between a first, resting position and a second, activated position; and
a control mechanism coupled to the holding shelf and capable of being activated so as to move the holding shelf between the resting position and the activated position,
wherein, in the resting position, the holding shelf is positioned to retain medical instruments within the medical instrument holding container, and
wherein, in the activated position, the holding shelf is positioned such that the medical instruments are caused to be released from the holding shelf and travel into the medical instrument disposal bin.

18. The medical instrument disposal system of claim 17, further comprising:
a bracket substantially surrounding at least the medical instrument holding container and the medical instrument disposal bin, wherein the bracket includes a sensor for identifying medical instruments positioned adjacent to the sensor.

19. The medical instrument disposal system of claim 17, wherein the sensor includes at least one of: a camera, a laser, a bar code scanner, an optical sensor, active light sensor, or load sensor.

20. The medical instrument disposal system of claim 17, wherein the sensor is communicatively coupled to an electronic medical records system.
